(19) United States
(12) Patent Application Publication
(10) Pub. No.: US 2007/0178054 A1
(43) Pub. Date: Aug. 2, 2007

(54) ORAL ANTIMICROBIAL COMPOSITION

(76) Inventors: Madhyastha Srinivasa, Winnipeg (CA); Karen LoVetri, Winnipeg (CA)

Correspondence Address: MERCHANT & GOULD PC, P.O. BOX 2903, MINNEAPOLIS, MN, 55402-0903, US

(21) Appl. No.: 11/558,184

(22) Filed: Nov. 9, 2006

Related U.S. Application Data
(60) Provisional application No. 60/735,788, filed on Nov. 9, 2005.
(60) Provisional application No. 60/744,425, filed on Apr. 7, 2006.

(51) Int. Cl. A61K 8/96

(52) U.S. Cl. 424/50

(57) ABSTRACT

The present invention includes compositions and methods for inhibiting the growth and formation of biofilms. The compositions and methods can employ antimicrobial compounds and/or antimicrobial peptides. In an embodiment, a composition includes a combination of at least one antimicrobial compound and at least one CSP analogue or CSP. In an embodiment, a method to inhibit the growth and/or formation of an oral biofilm includes administering a composition comprising at least one antimicrobial compound and at least one CSP analogue or CSP.

### CROSS REFERENCE TO RELATED APPLICATIONS

This application claims the benefit under 35 U.S.C. § 19(e) of U.S. Provisional Application No. 60/735,788, filed Nov. 9, 2005, and U.S. Provisional Application No. 60/744,425, filed Apr. 7, 2006, the entire disclosures of which are hereby incorporated by reference.

### FIELD OF THE INVENTION

The present invention relates to oral compositions comprising antimicrobial agents that inhibit dental plaque and caries-associated Streptococcus mutans growth and biofilm formation.

### BACKGROUND

Caries and periodontal diseases are two of the most common chronic infectious diseases affecting humankind and are always associated with dental plaque formed as a biofilm on tooth surfaces. Dental plaque is produced by sequential attachment of a variety of bacteria, which is dependent on both species involved and the surface composition (Kawashima et al., Oral. Microbiol. Immunol. 18: 220-225, 2003). Oral streptococci and Actinomyces spp. are the first to appear on the surface of the teeth. Streptococci account for approximately 20% of the salivary bacteria, which include Streptococcus spp. such as Streptococcus mutans, Streptococcus sobrinus, Streptococcus sanguis, Streptococcus gordonii, Streptococcus oralis and Streptococcus mitis. Although four streptococci, S. mutans, S. sobrinus, S. sanguis and S. oralis are directly involved in the initiation of dental caries, S. mutans is considered to be a principal etiological agent of caries (Devulapalle et al., Carbohydr. Res. 339:1029-1034, 2004). As S. mutans has evolved to depend on a biofilm lifestyle for survival and persistence in the oral cavity combined with its role as an opportunistic pathogen, it has become the best-studied example of a biofilm-forming, disease-causing Streptococcus (Burne, R. A., J. Dent. Res. 77: 445-452, 1998).

Quorum sensing is a mearts of intercellular communication between bacterial cells (Davies et al., Science 280:226-227, 1998). This mechanism allows bacteria to control gene expression and respond to population density as a group. Thus, bacteria can optimize their physiology to adapt to environmental stimuli (Li et al., J. Bacteriol. 184:6333-6342, 2002). Furthermore, bacteria utilizing quorum sensing can behave as a collective, thereby S. mutans can better colonize hosts, evolve as a species, and respond to mechanical, physical, and chemical stresses (Li et al., 2002, J. Bacteriol.). Therefore, bacteria in biofilms have an increased resistance to antimicrobials and host defenses (Petersen et al., J. Bacteriol. 187:4392-4400, 2005). Many Streptococci use quorum-sensing systems to regulate several physiological processes, including the incorporation of foreign DNA, acid tolerance, biofilm formation, and virulence. In Streptococci, quorum-sensing systems consist primarily of a small competence-stimulating peptide (CSP) that is detected by neighboring cells via a histidine kinase/response regulator pair.

### SUMMARY OF THE INVENTION

The present invention includes compositions and methods for inhibiting growth and formation of biofilms. The compositions and methods can employ antimicrobial compounds and/or antimicrobial peptides.

In an embodiment, a composition includes a combination of at least one antimicrobial compound and at least one CSP analogue.

In another embodiment, a composition includes a combination of at least one antimicrobial compound and CSP.

In a further embodiment, a method to inhibit growth and/or formation of an oral biofilm includes administering a composition comprising at least one antimicrobial compound and at least one CSP analogue or CSP.

### BRIEF DESCRIPTION OF THE DRAWINGS

FIG. 1 shows the effect of E2 peptide (20 μg/ml), nisin (N) (80 μg/ml), and a combination of E2 peptide (20 μg/ml) and nisin (N) (80 μg/ml) on S. mutans growth and biofilm formation. A control of S. mutans grown in media without an antimicrobial was also included.

FIG. 2 shows the effect of E2 peptide (20 μg/ml), xylitol (X) (15.2 mg/ml), and a combination of E2 peptide (20 μg/ml) and xylitol (X) (15.2 mg/ml) on S. mutans growth and biofilm formation. A control of S. mutans grown in media without an antimicrobial was also included.

FIG. 3 shows the effect of E2 peptide (20 μg/ml), chlorhexidine (CHX) (0.154 μg/ml), and a combination of E2 peptide (20 [μg/ml) and chlorhexidine (CHX) (0.154 μg/ml) on S. mutans growth and biofilm formation. A control of S. mutans grown in media without an antimicrobial was also included.

FIG. 4 shows the effect of E2 peptide (20 μg/ml), triclosan (25 μg/ml), and a combination of E2 peptide (20 μg/ml) and triclosan (25 μg/ml) on S. mutans growth and biofilm formation. A control of S. mutans grown in media without an antimicrobial was also included.

FIG. 5 shows the effect of E2 peptide (20 μg/ml), citric acid (C.A.) (1.2 mg/ml), and a combination of E2 peptide (20 μg/ml) and citric acid (C.A.) (1.2 mg/ml) on S. mutans growth and biofilm fonnation. A control of S. mutans grown in media without an antimicrobial was also included.

FIG. 6 shows the effect of E2 peptide (20 μg/ml), oleanolic acid (O.A.) (2 μg /ml), and a combination of E2 peptide (20 μg/ml) and oleanolic acid (O.A.) (2 μg /ml) on S. mutans growth and biofilm formation. A control of S. mutans grown in media without an antimicrobial was also included.

FIG. 7 shows the effect of E2 peptide (20 μg/ml), lansoprazole (L) (0.1 mM), and a combination of E2 peptide (20 μg/ml) and lansoprazole (L) (0.1 mM) on S. mutans growth and biofilm formation. A control of S. mutans grown in media without an antimicrobial was also included.

FIG. 8 shows the effect of E2 peptide (20 μg/ml), epigallocatechin gallate (EGCg) (150 μg/ml), and a combination of E2 peptide (20 μg/ml) and epigallocatechin gallate (EGCg) (150 μg/ml) on S. mutans growth and biofilm formation. A control of S. mutans grown in media without an antimicrobial was also included.

FIG. 9 shows the effect of E2 peptide (20 μg/ml), sodium fluoride (S.F.) (600 μg/ml), and a combination of E2 peptide (20 μg/ml) and sodium fluoride (S.F.) (600 μg/ml) on S. mutans growth and biofilm formation. A control of S. mutans grown in media without an anti-caries agent was also included.

FIG. 10 shows the effect of E2 peptide (20 μg/ml), chitosan (C) (1 μg/ml), and a combination of E2 peptide (20 μg/ml) and chitosan (C) (1 μg/ml) on S. mutans growth and biofilm formation. A control of S. mutans grown in media without an antimicrobial was also included.

FIG. 11 shows the effect of E2 peptide (20 μg/ml), nisin (N) (80 μg/ml), and a combination of E2 peptide (20 μg/ml) and nisin (N) (80 μg/ml) on biofilm-embedded S. mutans. A control of S. mutans grown in media without an antimicrobial was also included.

FIG. 12 shows the effect of E2 peptide (20 μg/ml), chlorhexidine (CHX) (0.15 μg/ml), and a combination of E2 peptide (20 μg/ml) and chlorhexidine (CHX) (0.15 μg/ml) on biofilm-embedded S. mutans. A control of S. mutans grown in media without an antimicrobial was also included.

FIG. 13 shows the effect of E2 peptide (20 μg/ml), citric acid (C.A.) (1.2 mg/ml), and a combination of E2 peptide (20 μg/ml) and citric acid (CA) (1.2 mg/ml) on biofilm-embedded S. mutans. A control of S. mutans grown in media without an antimicrobial was also included.

FIG. 14 shows the effect of E2 peptide (20 μg/ml), lansoprazole (L) (37 μg/ml), and a combination of E2 peptide (20 μg/ml) and lansoprazole (L) (37 μg/ml) on biofilm-embedded S. mutans. A control of S. mutans grown in media without an antimicrobial was also included.

FIG. 15 shows the effect of E2 peptide (20 μg/ml), chitosan (C) (1 μg/ml), and a combination of E2 peptide (20 μg/ml) and chitosan (C) (1 μg/ml) on biofilm-embedded S. mutans. A control of S. mutans grown in media without an antimicrobial was also included.

FIG. 16 shows the effect of E2 peptide (20 μg/ml), sodium fluoride (S.F.) (800 μg/ml), and a combination of E2 peptide (20 μg/ml) and sodium fluoride (S.F.) (800 μg/ml) on biofilm-embedded S. mutans. A control of S. mutans grown in media without an anti-caries agent was also included.

FIG. 17 shows the effect of CSP (50 μg/ml), nisin (N) (80 μg/ml), and a combination of CSP (50 μg/ml) and nisin (N) (80 μg/ml) on biofilm-embedded S. mutans. A control of S. mutans grown in media without an antimicrobial was also included.

FIG. 18 shows the effect of CSP (40 μg/ml), chlorhexidine (CHX) (0.15 μg/ml), and a combination of CSP (40 μg/ml) and chlorhexidine (CHX) (0.15 μg/ml) on biofilm-embedded S. mutans. A control of S. mutans grown in media without an antimicrobial was also included.

FIG. 19 shows the effect of CSP (40 μg/ml), triclosan (T) (500 μg/ml), and a combination of CSP (40 μg/ml) and triclosan (T) (500 μg/ml) on biofilm-embedded S. mutans. A control of S. mutans grown in media without an antimicrobial was also included.

FIG. 20 shows the effect of CSP (40 μg/ml), citric acid (CA) (1200 μg/ml), and a combination of CSP (40 μg/ml) and citric acid (CA) (1200 μg/ml) on biofilm-embedded S. mutans. A control of S. mutans grown in media without an antimicrobial was also included.

FIG. 21 shows the effect of CSP (40 μg/ml), zinc citrate (ZC) (1200 μg/ml), and a combination of CSP (40 μg/ml) and zinc citrate (ZC) (1200 μg/ml) on biofilm-embedded S. mutans. A control of S. mutans grown in media without an antimicrobial was also included.

FIG. 22 shows the effect of CSP (40 μg/ml), sodium fluoride (S.F.) (250 μg/ml), and a combination of CSP (40 μg/ml) and sodium fluoride (S.F.) (250 μg/ml) on biofilm-embedded S. mutans. A control of S. mutans grown in media without an anti-caries agent was also included.

FIG. 23 shows the effect of CSP (40 μg/ml), oleanolic acid (OA) (1.5 μg/ml), and a combination of CSP (40 μg/ml) and oleanolic acid (OA) (1.5 μg/mil) on biofilm-embedded S. mutans. A control of S. mutans grown in media without an antimicrobial was also included

FIG. 24 shows the effect of CSP (40 μg/ml), lansoprazole (L) (37 μg/ml), and a combination of CSP (40 μg/ml) and lansoprazole (L) (37 μg/ml) on biofilm-embedded S. mutans. A control of S. mutans grown in media without an antimicrobial was also included.

FIG. 25 shows the effect of CSP (40 μg/ml), chitosan (C) (0.25 μg/ml), and a combination of CSP (40 μg/ml) and chitosan (C) (0.25 μg/ml) on biofilm-embedded S. mutans. A control of S. mutans grown in media without an antimicrobial was also included.

FIG. 26 shows the effect of CSP (25 μg/ml), nisin (N) (80 μg/ml) alone and in combination on biofilm-embedded S. mutans grown on hydroxyapitite disks. A control of S. mutans grown in media without an antimicrobial was also included.

FIG. 27 shows the effect of CSP (40 μg/ml), triclosan (T) (500 μg/ml) alone and in combination on biofilm-embedded S. mutans grown on hydroxyapitite disks. A control of S. mutans grown in media without an antimicrobial was also included.

FIG. 28 shows the effect of CSP (40 μg/ml), zinc citrate (ZC) (300 μg/ml) alone and in combination on biofilm-embedded S. mutans grown on hydroxyapitite disks. A control of S. mutans grown in media without an antimicrobial was also included.

### DETAILED DESCRIPTION

Definitions

The tenn “amino acid” is used in its broadest sense and is meant to include the naturally occurring L α-amino acids or residues. The commonly used one and three letter abbreviations for naturally occurring amino acids are used herein (Voet & Voet, Biochemistry, 2d ed., pp. 58-59, (1995), John Wiley & Sons, Inc., Somerset, N.J.). The term includes all D-amino acids as well as chemically modified amino acids such as amino acid analogs, naturally occurring amino acids that are not usually incorporated into proteins such as norleucine (e.g., Voet & Voet, pp. 67-69), and chemically synthesized compounds having properties known in the art to be characteristic of an amino acid. For example, analogs or mimetics of phenylalanine or proline, which allow the same confonnational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

The tenm “antimicrobial” refers to a compound or a composition that kills or inhibits the growth of microorganisms, including, but not limited to bacteria and yeasts.

The tenm “bacteriocin” refers to a family of ribosomally synthesized peptide antibiotics that are produced by bacteria (Kolter & Moreno, 1992, Annu. Rev. Microbiol. 46:141-163). Bacteriocins are categorized based on biochemical and genetic characteristics into four different classes. Lantibiotics are Class I bacteriocins and contain two modified amino acid residues, lanthionine and/or methyllanthionins. S. mutans also produces bacteriocins named “mutacins”. The mutacin molecules are also antimicrobial.

The term “biofilm formation” refers to the attachment of microorganisms to surfaces and the subsequent development multiple layers of cells.

The term “dental caries” refers to a localized destruction of tissues of a tooth by acid produced from bacterial degradation of fermentable sugars. The chief etiological agent of dental caries is S. mutans. Degradation of fermentable sugars by S. mutans on the tooth surface produces an acid that destroys oral tissues, and more particularly, enamel and dentin.

The tenn “dental plaque” is a general tenn for the diverse microbial community (predominantly bacteria) found on the tooth surface, embedded in a matrix of polymers of bacterial and salivary origin. Further, “dental plaque-associated S. mutans” refers to S. mutans that is a component of the dental plaque.

The term “endocarditis” refers to an infection of the endocardial surface of the heart, which may include one or more heart valves, the mural endocardium, or a septal defect.

The tenn “gingivitis” refers to inflammation of gingival tissue without loss of connective tissue.

The term “inhibition” refers to at least a decrease of dental plaque-associated bacterial (e.g., S. mutans) growth and biofilm formation.

The term “mammal” for purposes of treatment refers to any animal classified as a mammal, including humans, domestic and farm animals, and zoo, sports, or pet animals, such as dogs, horses, cats, cattle, pigs, sheep, etc. Preferably, the mammal is human.

The term “oral diseases” refers to diseases and disorders affecting the oral cavity or associated medical conditions. Oral diseases include, but are not limited to, dental caries; periodontal diseases (e.g., gingivitis, adult periodontitis, early-onset periodontitis, etc.); mucosal infections (e.g., oral candidiasis, herpes simplex virus infections, recurrent aphthous ulcers, etc.); oral and pharyngeal cancers; and precancerous lesions.

The term “peptide” refers to two or more amino acids chained together by a bond called a “peptide bond.”

The term “periodontal disease” refers to an inflammatory process of the gingival tissues and/or periodontal membrane of the teeth, resulting in a deep gingival sulcus, possibly producing periodontal pockets and loss of alveolar bone.

The term “periodontitis” refers to inflammation and loss of connective tissue of the supporting or surrounding structure of teeth with loss of attachment.

The term “prophylaxis” refers to at least preventing a condition associated with S. mutans occurring in a mammal, particularly when the mammal is found to be predisposed to having the condition but has not yet been diagnosed as having it.

The term “quorum sensing” refers to the control of gene expression in response to cell density. Bacterial cells communicate amongst the cells of the biofilm utilizing secreted signalling molecules. Typically, gram-negative bacteria utilize homoserine lactones and gram-positive bacteria utilize small peptides as effector signalling molecules.

The term “subject” refers to a living vertebrate such as mammal (preferably human) in need of treatment.

The term “therapeutically effective amount” refers to a quantity of a composition high enough to provide a significant positive modification of the subject's condition(s) to be treated. A “therapeutically effective amount” as used herein includes a prophylactic amount, for example, an amount effective for preventing or protecting against dental caries and related diseases, and symptoms thereof, and amounts effective for alleviating or healing dental caries, related diseases, and symptoms thereof. By administering a peptide suitable for use in methods of the invention concurrently with an antimicrobial, the peptide and/or the antimicrobial may be administered in a dosage amount that is less than the dosage amount required when the antimicrobial is administered as a sole active ingredient. By administering lower dosage amounts of active ingredient, side effects associated therewith could be reduced.

The term “treatment” refers to an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In regards to dental caries, “treating or treatment” is intended to mean at least the mitigation of a condition associated with S. mutans in a subject, such as a mammal, including but not limited to, a human, that is affected at least in part by the condition, and includes, but is not limited to, modulating, inhibiting the condition, and/or alleviating the condition.

Competence-Stimulating Peptide (CSP)

Many Streptococci use quorum-sensing systems to regulate several physiological processes, including incorporation of foreign DNA, acid tolerance, biofilm formation, and virulence. S. mutans also utilizes quorum-sensing systems. The S. mutans quorum sensing system is mediated by a competence-stimulating peptide (CSP). This signal transduction system is encoded by the comCDE genes (Li et al., 2002, J. Bacteriol.). comC encodes a CSP precursor, comD encodes a histidine kinase that is the receptor for CSP, and comE encodes a response regulator. In most S. mutans strains (e.g., UA159, NG8, LT11, and GB14), comC encodes a 46 amino acid precursor of the following sequence:

MKKTLSLKNDFKEIKTDELEIIIGGSGSLSTFFRLFNRSFTQALGK (SEQ ID NO: 1). However, strain BM71 has a L5P substitution. To form mature CSP, a precursor is cleaved after 25 amino acids to form mature CSP of the following sequence: SGSLSTFFRLFNRSFTQALGK (SEQ ID NO: 2). S. mutans strain JH1005 has a one amino acid substitution and a 3 amino acid truncation at the carboxyl terminal for the following sequence: SGTLSTFFRLFNRSFTQA (SEQ ID NO: 3).

Deletion or inactivation of any of the comCDE genes produces an abnormal S. mutans biofilm, either an altered biofilm architecture or a decrease in biomass. CSP also regulates the smb operon (Yonezawa & Kuramitsu, Antimicrob. Agents Chernother. 49: 541-548, 2005). The smb operon encodes a class I bacteriocin family lantibiotic. Disruption of the smb operon thereby blocks the production of the lantibiotic. Thereby, S. mutans cannot initiate or sustain colonization in the biofilm (Rogers et al., 1979, Infect. Immun. 23: 571-576; van der Hoeven et al., 1979, Infect. Immun. 23: 2314-2316). Inhibition of CSP signalling will not allow for full S. mutans colonization, growth, and virulence. Therefore, in an embodiment, inhibition of CSP signalling provides a mechanism to treat subjects with an oral biofilm, subjects with S. mutans associated dental plaque, or subjects with dental caries.

### Compositions

The present invention includes enhanced oral antimicrobial compositions for the prevention or prophylaxis of oral diseases and endocarditis comprising at least one peptide analogue of S. mutans CSP or CSP itself. The peptide analogues (Table 1) comprise F1 (SEQ ID NO: 4), F2 (SEQ ID NO: 5), HI (SEQ ID NO: 6), H2 (SEQ ID NO: 7), B2 (SEQ ID NO: 8), C2 (SEQ ID NO: 9), E2 (SEQ ID NO: 10), and B3 (SEQ ID NO: 11). In a preferred embodiment of the invention, the compositions are prepared using the E2 (SEQ ID NO: 10) peptide or CSP (SEQ ID NO: 2).

TABLE 1
Synthetic CSP analogues

| | | |
|---|---|---|
| Peptide | Amino acid sequence | SEQ ID NO: |

| | | |
|---|---|---|
| F1 | SGSLSTFFRLFNRSFTQALK | 4 |
| F2 | SGSLSTFFRLFNRSFTQALGV | 5 |
| H1 | SGSLSTFFRLFNRSFTQLGK | 6 |
| H2 | SGSLSTFFVLFNVSFTQALGV | 7 |
| B2 | SGSLSTPFVLFNRSFTQALGK | 8 |
| C2 | SGSLSTFFALFNRSFTQALGK | 9 |
| E2 | SGSLSTFFRLFNASFTQALGK | 10 |
| B3 | SGTLSTFFRLFNRSFTQA | 11 |

In an embodiment, a CSP analogue-containing composition includes an antimicrobial compound. A CSP analogue in combination with an antimicrobial compound has an enhanced inhibitory effect on S. mutans growth and biofilm fonnation. Furthermore, addition of an antimicrobial compound to a composition containing CSP analogue can make the composition effective against other oral pathogens associated with dental caries and periodontal diseases.

In another embodiment, a CSP-containing composition includes an antimicrobial compound. CSP in combination with an antimicrobial compound has an enhanced inhibitory effect on S. mutans growth. Furthermore, addition of an antimicrobial compound to a composition containing CSP can make the composition effective against other oral pathogens associated with dental caries and periodontal diseases.

In an embodiment of the invention, an enhanced oral antimicrobial composition comprises CSP or at least one CSP analogue and one or more antimicrobial agents comprising benzimidazoles (e.g., lansoprazole and omeprazole), polyols (e.g., xylitol, sorbitol, etc.), polyphenols (e.g., epigallocatechin gallate), antiseptics (e.g., triclosan, chlorhexidine salt, cetylpyridinium chloride, etc.), antibiotics, anti-caries agents, and bacteriocins (e.g., nisin, epidermin, gallidennin, cinnamycin, duramycin, lacticin 481, etc.). Additionally, the oral compositions may comprise ingredients such as citrate (e.g., citric acid, zinc citrate, sodium citrate, etc.), triterpenoids (e.g., oleanolic acid and ursolic acid) and chitosan

In an embodiment, a composition comprises a benzimidazole and at least one CSP analogue or CSP. Compounds which inhibit the gastric H⁺/K⁺-ATPase enzyme are generally known as “proton pump inhibitors” (PPI). Some of the PPIs capable of inhibiting the gastric H⁺/K⁺-ATPase enzyme include the substituted benzimidazoles lansoprazole (U.S. Pat. No. 4,628,098), omeprazole (U.S. Pat. Nos. 4,255,431 and 5,693,818), pantoprazole (U.S. Pat. No. 4,758,579), and raberprazole (U.S. Pat. No. 5,045,552), which are hereby incorporated by reference. Diseases currently treated by PPIs and specifically by the four above-mentioned drugs include peptic ulcer, heart burn, reflux esophagitis, errosive esophagitis, non-ulcer dispepsia, infection by Helicobacter pylori, and asthma among others.

In an embodiment, a composition comprises an antibiotic and CSP or at least one CSP analogue. Antibiotics are well known. Groups of antibiotics include, but are not limited to, β-lactam inhibitors (e.g., penicillin, ampicillin, amoxicillin, methicillin, etc.), cephalosporins (e.g., cephalothin, cephamycin, etc.), aminoglycosides (e.g., streptomycin, tobramycin, etc.), polyenes (e.g., amphotericin, nystatin, etc.), macrolides (e.g., erythomycin, etc.), tetracyclines (e.g., tetracycline, doxycycline, etc.), nitroimidazole (e.g., metronidazole), quinolones (e.g., nalidixic acid), rifamycins (e.g., rifampin), and sulfonamides (e.g., sulfanilamide), nitroaromatics (e.g., chloramphenicol) and pyridines (e.g., isoniazid).

In an embodiment, a composition comprises a polyphenol and CSP or at least one CSP analogue. An example of a polyphenol is epigallocatechin gallate (EGCg). EGCg is a catechin isolated from green tea and has anti-oxidant and immunomodulatory activities (Matsunaga et al., 2002, Clin. Diagn. Lab. Immunol. 9: 864-871). Antimicrobial activity of polyphenols such as tannins from thyme, cashew and eucalyptus has also been reported (Cowan, Clin. Microbiol. Rev. 12:564-582, 1999)

In an embodiment, a composition comprises a polyol and CSP or at least one CSP analogue. Polyols, also known as sugar alcohols, are carbohydrate sugar-free sweetners. Polyols are derived from carbohydrates with carbonyl groups reduced to a primary or secondary hydroxyl group. Polyols include, but are not limited to, sorbitol, xylitol, mannitol, and maltitol. S. mutans can ferment polyols to a limited extent. Theoretically, polyols produce a negative energy cycle in which S. mutans loses energy without producing acids. A negative energy cycle would also limit growth and/or biofilm formation.

In an embodiment, a composition comprises a bacteriocin and CSP or at least one CSP analogue. Bacteriocins include lantibiotics. S. mutans produces bacteriocin antimicrobial molecules called mutacins. Mutacins have been classified into two families: the lantibiotics and the non-antibiotics. Based on the mutacin's bactericidal activities, sensitivities to other or self-produced mutacins, and the presence of plasmids, mutacins are classified into types, I, II, III, and IV. Mutacins I, II, and III are classified as lantibiotics, and mutacin IV is a dipeptide non-lantibiotic bacteriocin. Examples of bacteriocins include, but are not limited to, nisin, epidernin, gallidermin, cinnamycin, duramycin, lacticin 481, mutacin I, B-Ny266, and mutacin 1140. See, also, U.S. Patent Nos. 6,699,970; 6,699,839; 6,475,771; 6,391,285; 6,342,385; 6,218,362; and 5,932,469.

In an embodiment, a composition comprises an antiseptic and CSP or at least one CSP analogue. Antiseptics are agents that kill or inhibit the growth of microorganisms on the external surfaces of the body. Antiseptics include, but are not limited to, triclosan, chlorhexidine salt, and cetylpyridinium chloride.

In an embodiment, a composition comprises one or more anti-caries agents and CSP or at least one CSP analogue. Various anti-caries agents are well known and are included in an embodiment of the present invention. Various anti-caries agents include, but are not limited to benzoic esters, sesquiterpene alcohols (e.g., farnesol, nerolidol, bisabolol, and santalol), halogenated carbanilides, phenolic compounds, aromatic halophenols, resorcinols, catechols, bisphenolic compounds, histidine-rich polypeptides, fluorides (sodium fluoride, stannous fluoride, amine fluorides, monosodiumfluorophosphate, calcium lactate, calcium glycerophosphate, proline-rich proteins, non-immunogenic amino acid segment, and antibodies of S. mutans.

In a further embodiment of the invention, a composition comprises between 1 μg/ml and 200 μg/ml of a CSP analogue or CSP and between 0.15 μg/ml and 15 mg/ml of a benzimidazole, a polyol, a polyphenol, an antiseptic, an antibiotic, a bacteriocin, a citrate, or a triterpenoid. In further embodiments of the invention, the composition can comprise between 1 μg/ml and 100 μg/ml, 1 μg/ml and 50 g/ml, 10 μg/ml and 200 μg/ml o, or 100 μg/ml and 200 μg/ml of a CSP analogue or CSP in combination with a benzimidazole, a polyol, a polyphenol, an antiseptic, an antibiotic, a bacteriocin, a citrate, or a triterpenoid. In further embodiments of the invention, the composition can comprise between 0.5 μg/ml and 15 mg/ml, 1.0 μg/ml and 15 mg/ml, 10 μg/ml and 15 mg/ml, 100 μg/ml and 15 mg/ml, 500 μg/ml and 15 mg/ml, 1.0 mg/ml and 15 mg/ml, 10 mg/ml and 15 mg/ml, 0.15 μg/ml and 10 mg/ml, 0.15 μg/ml and 1.0 mg/ml, 0.15 μg/ml and 500 μg/ml, 0.15 μg/ml and 250 μg/ml, 0.15 μg/ml and 200 μg/ml, 0.15 μg/ml and 100 μg/ml, 0.15 μg/ml and 50 μg/ml, 0.15 μg/ml and 10 μg/ml, 0.15 μg/ml and 5 μg/ml, 0.15 5 μg/ml and 1.0 μg/ml, 0.15 μg/ml and 0.5 μg/ml, 1.0 μg/ml and 500 μg/ml, 5 μg/ml and 500 μg/ml, 10 μg/ml, and 500 μg/ml, 50 μg/ml and 500 μg/ml, 100 μg/ml and 500 μg/ml, 250 μg/ml and 500 μg/ml, 1.0 μg/ml and 200 μg/ml, 1.0 μg/ml and 100 μg/ml, or 10 μg/ml and 100 μg/ml of a benzimidazole, a polyol, a polyphenol, an antiseptic, an antibiotic, a bacteriocin, a citrate, or a triterpenoid in combination with a CSP analogue or CSP.

In an embodiment, a composition is effective for inhibiting S. mutans growth and biofilm fonnation, which employs a quorum sensing system. S. mutans is a resident of the biofilm environment of dental plaque (oral biofilm). Under appropriate environmental conditions, populations of S. mutans and the pH of the surrounding plaque will drop. S. mutans, being among the most acid tolerant organisms residing in dental plaque, will increase its numbers in this acidic environment and eventually become a dominant member of the plaque community. This situation eventually leads to dissolution of the tooth enamel, resulting in the development of dental caries. Other oral streptococci, include, but are not limited to Streptococcus sobrinius, Streptococcus sanguis, Streptococcus gordonii, Streptococcus oralis and Streptococcus mitis. Infections can be modulated using embodiments of the invention.

An embodiment of the invention may also include other pharmaceutically acceptable vehicles, diluents, and additives such as antioxidants, buffers and solutes, which render the formulation isotonic in the intended recipient; and aqueous and non-aqueous sterile suspensions which may include suspending agents and thickening agents.

Oral Formulations

A composition of the invention can be added to a variety of formulations suitable for delivery of the composition to the oral cavity, including, but not limited to, mouthwash solutions, abrasive dentifrice gels, denture washes, nonabrasive dentifrice gels, denture washes or soaks, denture adhesives or cements, chewing gums, candies, soft drinks, and sports drinks. In order to provide such formulations, a composition of this invention is combined with one or more orally acceptable carriers and/or excipients.

Fonnulations including, but not limited to, mouthwash solutions, abrasive dentifrice gels, denture washes, nonabrasive dentifrice gels, denture washes or soaks, denture adhesives or cements, chewing gums, candies, soft drinks, sports drinks and other orally acceptable compositions comprising a CSP analogue in combination with a benzimidazole, a polyol, a polyphenol, an antiseptic, an antibiotic, a bacteriocin, a citrate, or a triterpenoid or chitosan can be prepared by any known method.

In general, methods of manufacturing oral antimicrobial compositions comprise combining an orally acceptable carrier and an effective amount of CSP or its analogue with a benzimidazole, a polyol, a polyphenol, an antiseptic, an antibiotic, a bacteriocin, an anti-caries agent, a citrate, a triterpenoid, or chitosan.

A variety of carriers and excipients can be used to formulate an embodiment of this invention and are well known. Such orally acceptable vehicles include, but are not limited to, water, ethanol, humectants such as polypropylene glycol, glycerol and sorbitol, gelling agents such as cellulose derivatives, polyoxypropylene/polyoxyethylene block copolymers, binding agents such as Gantrez®, pyrophosphates, bisphosphates, thickening agents such as Carbopol® 934, gel stabilizers such as silicon dioxides, sweeteners such as sodium saccharin, and other approved flavors, preservatives such as sodium benzoate, potassium sorbate, methyl and ethyl parabens, detergents such as sodium lauryl sulfate, sodium lauryl sarcosinate and approved colors.

Method of Treatment

Another aspect of this invention includes a method for treating dental caries, infective endocarditis, and periodontal diseases. In general, dental caries and periodontal diseases may be treated by contacting the oral cavity of a subject with an amount of CSP or a CSP analogue in combination with one or more anti-caries/antimicrobial agents effective to reduce S. mutans and other oral bacteria associated with dental plaque. In one embodiment, CSP or its analogue is formulated as an orally acceptable medicament as described herein comprising a carrier and an effective amount of composition comprising CSP or its analogue as an active ingredient.

With respect to dosage of CSP or its analogue, whether alone or in combination with one or more additional anti-caries/antimicrobial agents, a therapeutically effective amount can vary with the condition to be treated, its severity, the treatment regime to be employed, the phannacokinetics of the agent used, as well as the subject (animal or human) treated.

An exemplary dosing regime of an oral composition of this invention is application of a composition to the oral cavity of a subject every time a subject eats a food containing sugar. For example, people generally eat foods containing sugar from one to three times a day. According to this embodiment, a subject would apply a composition of the invention to the oral cavity from one to three times daily soon after consuming a sugar-containing food or beverage as part of a routine oral hygiene program to inhibit or treat dental caries, as a routine to prevent or treat gingivitis, or as a routine to prevent or treat endocarditis.

In a further embodiment of the invention, an enhanced oral antimicrobial composition does not present a significant tooth-staining problem.

The present invention may be better understood with reference to the following examples. These examples are intended to be representative of specific embodiments of the invention, and are not intended as limiting the scope of the invention.

### EXAMPLES

Bacterial Strains and Growth Conditions

Streptococcus mutans UA159 strain was used in these Examples. S. mutans strain UA159 was grown in Todd-Hewitt broth containing 0.3% yeast extract (THYE) at pH 7.0 and was subcultured routinely on THYE agar plates and incubated at 37° C. in an anaerobic chamber (5% CO₂). In liquid media, cultures were incubated in closed screw-cap tubes without agitation at 37° C. in an anaerobic chamber (5% CO₂)

Synthesis of CSP and CSP Analogues

Competence stimulating peptide (CSP) and its analogues were synthesized based on the sequence of the mature 21 amino acid CSP (SGSLSTFFRLFNRSFTQALGK; SEQ ID NO: 2). The CSP peptide analogues (F1, F2, H1, H2, B2, C2, E2 and B3) were synthesized by the Advanced Protein Technology Centre, Peptide Synthesis Facility of Hospital for Sick Children (Toronto, ON) and Mimotopes (Roseville, Minn.). The F1 and H1 analogues were generated by deleting the 2^{nd} and 4^{th} residues from the C′ termini, separately. While lysine was substituted with valine in F2, arginine and lysine were substituted with valine in H2 analogue. In B2 and C2 analogues, the charged residues were substituted with neutral (alanine) or hydrophobic (valine) residues. In E2 analogue, second arginine (from the C′ terminus) was substituted with neutral alanine. The B3 analogue was generated by substituting 3^{rd} residue from the N′ terminus with threonine and by deleting 1^{st}, 2^{nd} and 3^{rd} residues from the C′ terminus. The sequences of CSP analogues are listed in the TABLE 1.

### Example 1

### Effects of Compositions Comprising E2 Peptide and Antimicrobial Agents on S. mutans Growth and Biofilm Formation

An in vitro assay was performed to detennine whether E2 analogue of CSP in combination with antimicrobial agents such as nisin, chlorhexidine, triclosan, oleanolic acid, lansoprazole, xylitol, epigallocatechin gallate, citrate, chitosan and sodium fluoride (an anti-caries agent) would show enhanced inhibitory effects on S. mutans growth and biofilm formation.

Bioflm Assay

Biofilm formation by S. mutans UA159 was assayed and quantified using a slightly modified method described previously (Li et al., J. Bacteriol. 184: 2699-2708). The growth of biofilms on a 96-well polystyrene microtiter plate was initiated by inoculating 10 μl of an overnight S. mutans culture (1.2×10⁷ CFU/ml) into 300 μl of semi-defined minimal medium (58 mM K₂HPO₄, 15 mM KH₂PO₄, 10 mM (NH₄)₂SO₄, 35 mM NaCl, and 2 mM MgSO₄.7H₂O) supplemented with filter-sterilized vitamins (0.04 mM nicotinic acid, 0.1 mM pyridoxine HCl, 0.1 mM pantothenic acid, 1 mM riboflavin, 0.3 μM thiamine HCl, 0.05 μM D-biotin), amino acids (4 mM L-glutamic acid, 1 mM L-arginine HCl, 1.3 mM L-cysteine HCl, 0.1 mM L-tryptophan), 0.2% casamino acids, and 20 mM glucose containing E2 peptide (0 and 5 μg/ml) in the individual wells of a 96-well microtiter plate. Wells without cells were used as blank controls.

The microtiter plates were then incubated at 37° C. in an anaerobic chamber (5% CO₂) for 24 hours without agitation. After the incubation, the growth was measured at 600 nm with a microplate reader. The planktonic cells were carefully removed, and plates were air dried overnight. The plates were then stained with 0.4% crystal violet for 10 minutes, rinsed with sterile distilled water and air dried for 15 minutes. Biofilm was quantified by measuring the absorbance of stained biofilm at 630 nm with a microplate reader.

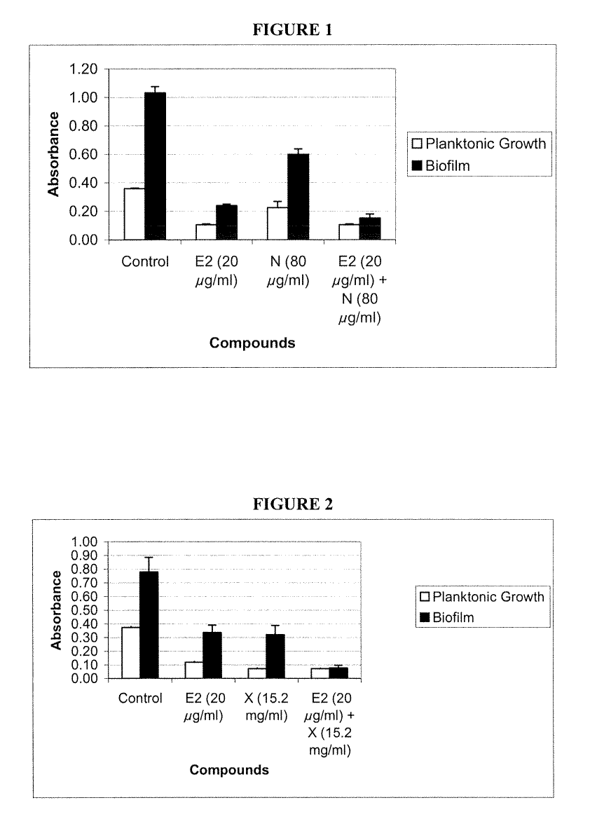

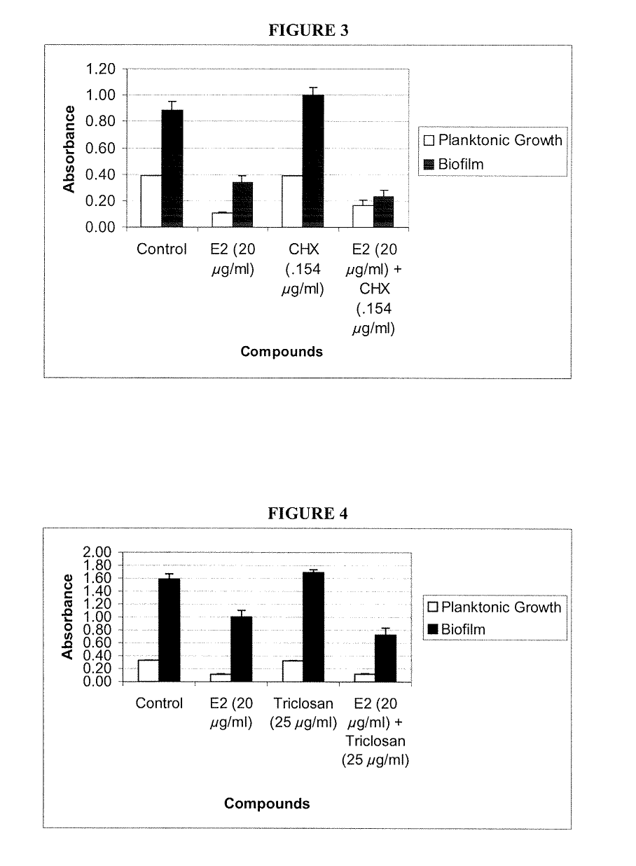

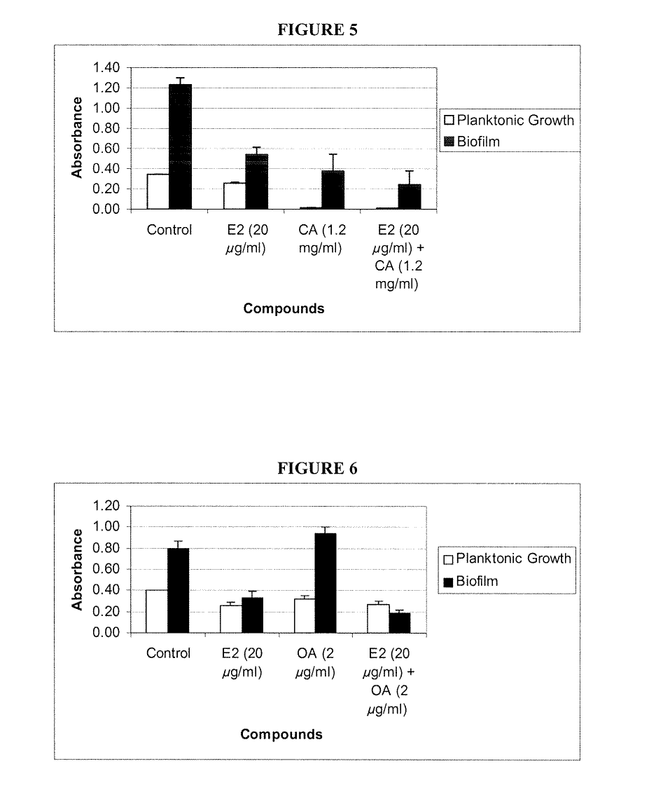

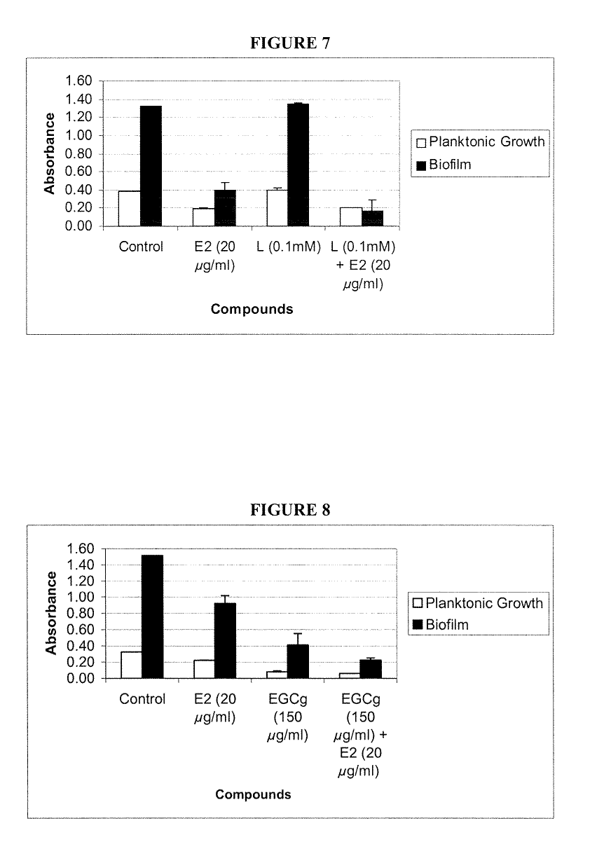

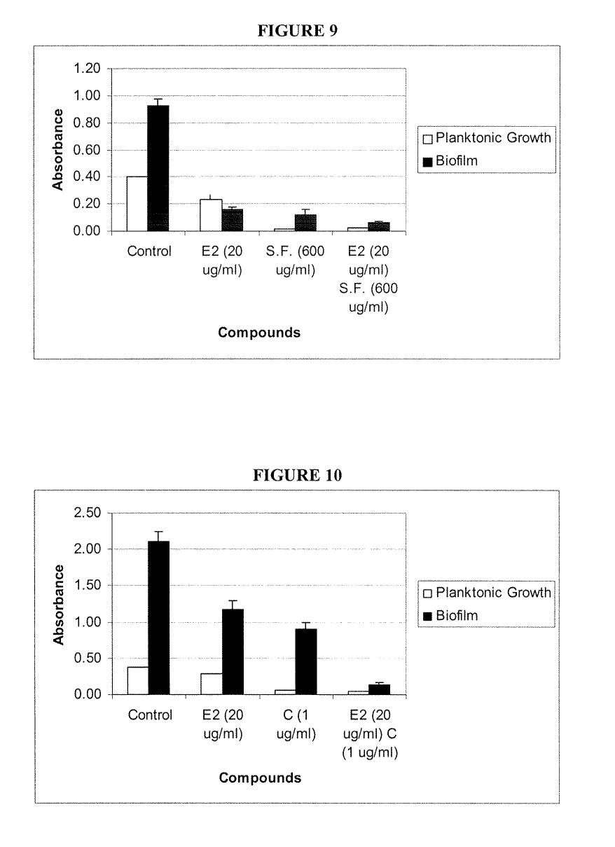

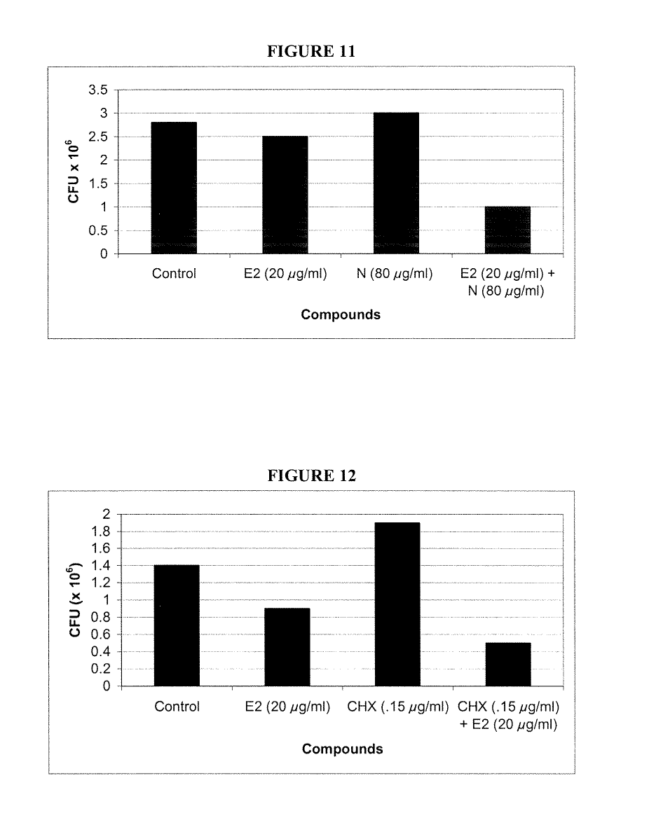

Results E2 peptide (20 μg/ml) in combination with the above antimicrobial compounds (concentrations ranged from 0.15 fig to 15.2 mg per ml) showed enhanced inhibitory effects on S. mutans growth as well as biofilm formation (Tables 2a and 2b). The percent inhibition of growth and biofilm formation varied from 70 to 100% and 60 to 90%, respectively. The results of Table 2 are graphically depicted in FIGS. 1-10.

TABLE 2a
Effects of compositions comprising E2 peptide and
antimicrobial agents on S. mutans growth and biofilm formation^{1,2}

| | | | Absorbance (OD) | | | |
|---|---|---|---|---|---|---|
| Compound | Concentration | | Growth (OD @ 600 nm) | | Biofilm (OD @ 630 nm) | |
| Control | 0 | | 0.36 | (0) | 1.03 | (0.04) |
| E2 | 20 | μg/ml | 0.11 | (0.01) | 0.24 | (0.01) |
| Nisin | 80 | μg/ml | 0.23 | (0.04) | 0.6 | (0.04) |
| E2 + Nisin | | | 0.11 | (0.01) | 0.15 | (0.03) |
| Control | 0 | | 0.37 | (0.01) | 0.78 | (0.11) |
| E2 | 20 | μg/ml | 0.12 | (0.01) | 0.34 | (0.05) |
| Xylitol | 15.2 | mg/ml | 0.07 | (0.01) | 0.32 | (0.07) |
| E2 + Xylitol | | | 0.07 | (0) | 0.08 | (0.02) |
| Control | 0 | | 0.39 | (0) | 0.88 | (0.07) |
| E2 | 20 | μg/ml | 0.11 | (0.01) | 0.34 | (0.05) |
| CHX | 0.154 | μg/ml | 0.39 | (0) | 1.00 | (0.06) |
| E2 + CHX | | | 0.17 | (0.04) | 0.24 | (0.04) |
| Control | | | 0.33 | (0) | 1.58 | (0.08) |
| E2 | 20 | μg/ml | 0.12 | (0.01) | 1.00 | (0.1) |
| Triclosan | 25 | μg/ml | 0.33 | (0) | 1.69 | (0.05) |
| E2 + Triclosan | | | 0.12 | (0.01) | 0.73 | (0.11) |
| Control | 0 | | 0.34 | (0) | 1.23 | (0.07) |
| E2 | 20 | μg/ml | 0.26 | (0.01) | 0.54 | (0.07) |
| Citric acid | 1.2 | mg/ml | 0.02 | (0) | 0.38 | (0.17) |
| E2 + Citric acid | | | 0.01 | (0) | 0.24 | (0.13) |

¹Values represent averages of at least three determinations (n = 3) with standard deviation in
parentheses.
²E2 = E2 peptide, CHX = Chlorhexidine, OA = Oleanolic acid, EGCg = Epigallocatechin gallate.

TABLE 2b
Effects of compositions comprising E2 peptide and
antimicrobial agents on S. mutans growth and biofilm formation^{1,2}

| | | | Absorbance (OD) | | | |
|---|---|---|---|---|---|---|
| Compound | Concentration | | Growth (OD @ 600 nm) | | Biofilm (OD @ 630 nm) | |
| Control | 0 | | 0.4 | (0) | 0.8 | (0.07) |
| E2 | 20 | μg/ml | 0.26 | (0.03) | 0.33 | (0.06) |
| OA | 2 | μg/ml | 0.32 | (0.02) | 0.94 | (0.07) |
| E2 + OA | | | 0.27 | (0.04) | 0.19 | (0.03) |
| Control | 0 | | 0.38 | (0) | 1.32 | |
| E2 | 20 | μg/ml | 0.2 | (0) | 0.4 | (0.08) |
| Lansoprazole | 0.1 | mM | 0.4 | (0.02) | 1.35 | (0.01) |
| E2 + Lansoprazole | | | 0.2 | (0) | 0.17 | (0.12) |
| Control | 0 | | 0.32 | (0) | 1.52 | |
| E2 | 20 | μg/ml | 0.22 | (0) | 0.92 | (0.1) |
| EGCg | 150 | μg/ml | 0.08 | (0.01) | 0.41 | (0.14) |
| E2 + EGCg | | | 0.06 | (0) | 0.23 | (0.03) |
| Control | 0 | | 0.40 | (0) | 0.93 | (0.04) |
| E2 | 20 | μg/ml | 0.23 | (0.04) | 0.16 | (0.01) |
| Sodium fluoride³ | 600 | μg/ml | 0.02 | (0) | 0.12 | (0.04) |
| E2 + Sodium fluoride | | | 0.02 | (0) | 0.07 | (0.01) |
| Control | 0 | | 0.37 | (0) | 2.10 | (0.14) |
| E2 | 20 | μg/ml | 0.28 | (0.01) | 1.18 | (0.11) |
| Chitosan | 1 | μg/ml | 0.05 | (0.01) | 0.90 | (0.10) |
| E2 + Chitosan | | | 0.04 | (0.01) | 0.13 | (0.03) |

¹Values represent averages of at least three determinations (n = 3) with standard deviation in parentheses.
²E2 = E2 peptide, CHX = Chlorhexidine, OA = Oleanolic acid, EGCg = Epigallocatechin gallate.
³An anti-caries agent

### Example 2

### Effects of Compositions Comprising E2 Peptide and Antimicrobial Agents on the Survival of Biofilm-Embedded S. mutans

An in vitro assay was perfonmed to determine whether E2 peptide in combination with nisin, chlorhexidine, citric acid, lansoprazole, chitosan and sodium fluoride would show enhanced inhibitory effects on the survival of biofilm-embedded S. mutans.

Assay for Biofilm-Embedded S. mutans

Biofilms were developed on 12-well polystyrene microtiter plates to provide a rapid and simple method for assaying biofilm-embedded live oral bacteria (e.g. S. mutans). A 4× diluted THYE medium supplemented with final concentration of 0.01% hog gastric mucin (Sigma, St. Louis, Mo.) was used as biofilm medium (BM). Formation of biofilms was initiated by inoculating 20 μl of S. mutans cell suspension (1.2×10⁷ CFU/ml) into each well containing 2 ml of BM and four wells were set up: two for control and two for treatment with compositions comprising synthetic E2 peptide and Nisin or E2 and CHX. After cultures were incubated at 37° C. for 20 hours under an anaerobic condition, fluid medium was removed. The wells were rinsed once with 10 mM PBS buffer (pH 7.2) and biofilm-embedded cells were collected in two ml PBS buffer, gently sonicated for 15 seconds, serially diluted, spread on THYE plates, and incubated at 37° C. under anaerobic conditions. Biofilm-embedded viable cells were quantified by colony forming unit (CFU) counts after 48 hours of incubation.

Results

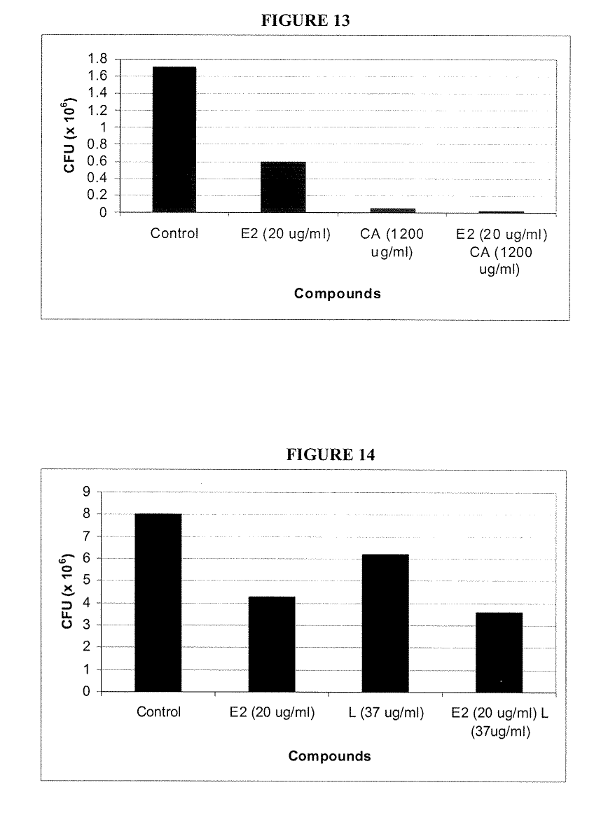

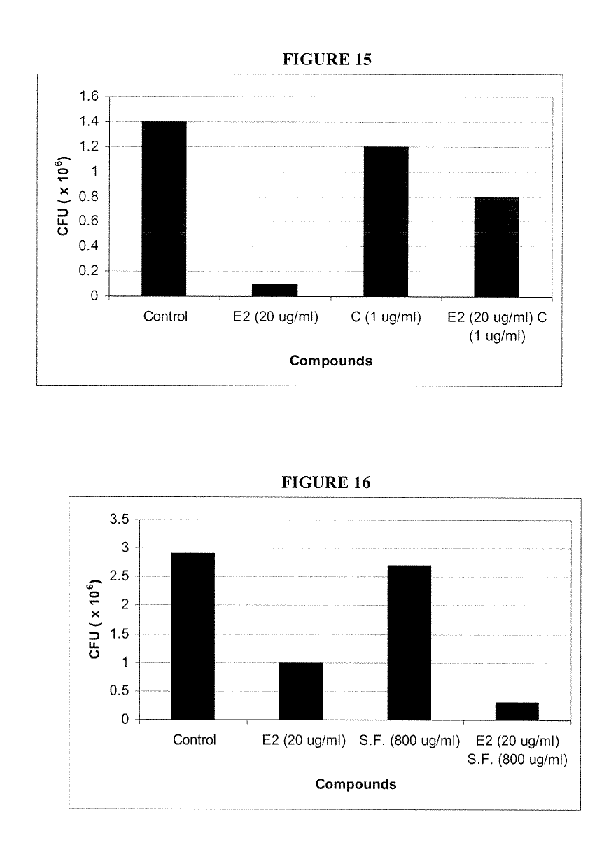

E2 peptide in combination with the above mentioned antimicrobials and anti-caries compounds showed an enhanced inhibitory effect on the survival of biofilm-embedded S. mutans as detennined by viable colony fonning unit (CFU) counts (Table 3). The results of Table 3 are graphically depicted in FIGS. 11-16. The combination of E2 with either nisin or chlorhexidine or citric acid or lansoprazole or sodium fluoride had more than an additive effect in decreasing the number of CFU.

TABLE 3
Effects of compositions comprising E2 Peptide and antimicrobial
agents on Biofilm-Embedded S. mutans¹

| Compound | Concentration (μg/ml) | CFU × 10⁶ |
|---|---|---|
| Control | 0 | 2.8 |
| E2 | 20 | 2.5 |
| Nisin | 80 | 3.0 |
| E2 + Nisin | | 1.0 |
| Control | 0 | 1.4 |
| E2 | 20 | 0.9 |
| Chlorhexidine | 0.15 | 1.9 |
| E2 + Chlorhexidine | | 0.5 |
| Control | 0 | 1.7 |
| E2 | 20 | 0.6 |
| Citric acid | 1200 | 0.05 |
| E2 + Citric acid | | 0.02 |
| Control | 0 | 8 |
| E2 | 20 | 4.3 |
| Lansoprazole | 37 | 6.2 |
| E2 + Lansoprazole | | 3.6 |
| Control | 0 | 1.4 |
| E2 | 20 | 0.1 |
| Chitosan | 1 | 1.2 |
| E2 + Chitosan | | 0.8 |
| Control | 0 | 2.9 |
| E2 | 20 | 1 |
| Sodium fluoride² | 800 | 2.7 |
| E2 + Sodium fluoride | | 0.3 |

¹Values represent averages of at least three determinations, E2 = E2 peptide, CFU = colony forming units.
²An anti-caries agent

### Example 3

### Effects of Compositions Comprising CSP and Antimicrobial Agents on the Survival of Biofllm-Embedded S. mutans

An in vitro assay was performed to determine whether CSP in combination with nisin, chlorhexidine, triclosan, citric acid, zinc citrate, sodium fluoride, oleanolic acid, lansoprazole, or chitosan would show enhanced inhibitory effects on the survival of biofilm-embedded S. mutans.

Assay for Biofilm-Embedded S. mutans

Biofilms were developed in 12-well polystyrene microtiter plates to provide a rapid and simple method for assaying biofilm-embedded live oral bacteria (e.g. S. mutans). A 4× diluted THYE medium supplemented with final concentration of 0.01% hog gastric mucin (Sigma) was used as biofilm medium (BM). Fonnation of biofilms was initiated by inoculating 20 μl of S. mutans cell suspension (1.2×10⁷ CFU/ml) into each well containing 2 ml of BM and four wells were set up: two for control and two for treatment with compositions comprising synthetic CSP and Nisin or chlorhexidine or xylitol or triclosan or citric acid or zinc citrate or sodium fluoride or oleanolic acid or lansoprazole or epigallocatechin gallate or chitosan. After cultures were incubated at 37° C. for 20 hours under an anaerobic condition, fluid media were removed. The wells were rinsed once with 10 mM PBS buffer (pH 7.2) and biofilm-embedded cells were collected in two ml PBS buffer, gently sonicated for 15 seconds, serially diluted, spread on THYE plates, and incubated at 37° C. under anaerobic conditions. Biofilm-embedded viable cells were quantified by colony forming unit (CFU) counts after 48 hours of incubation.

Results

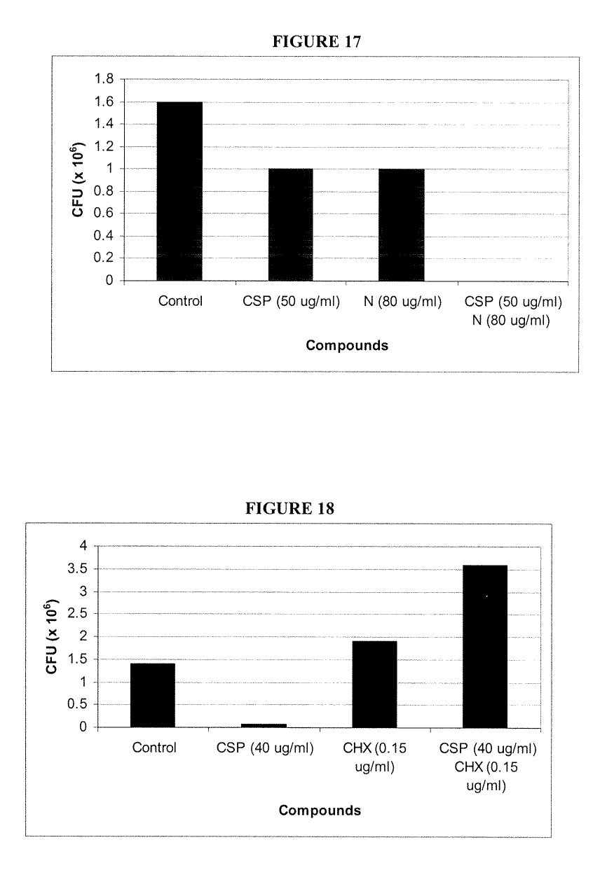

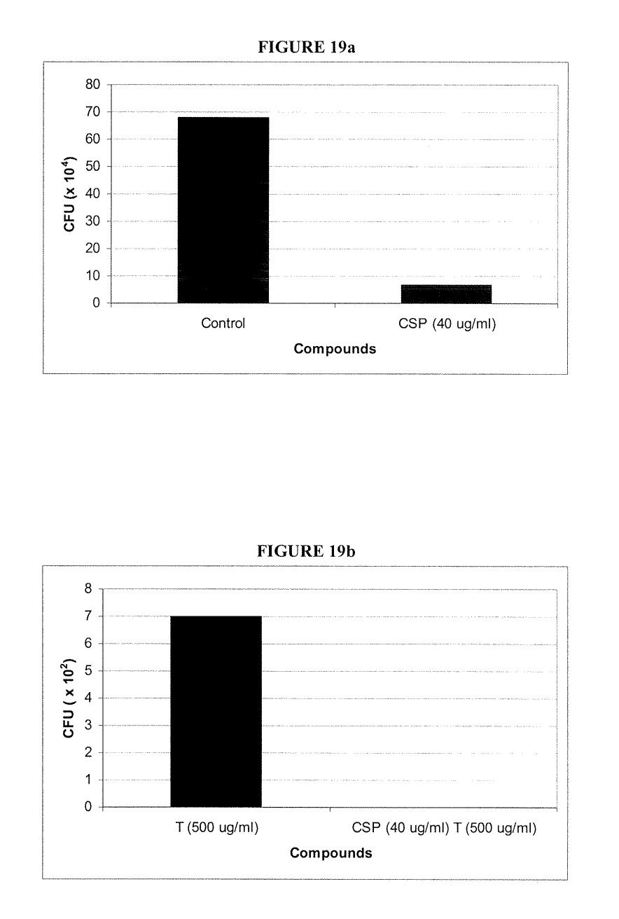

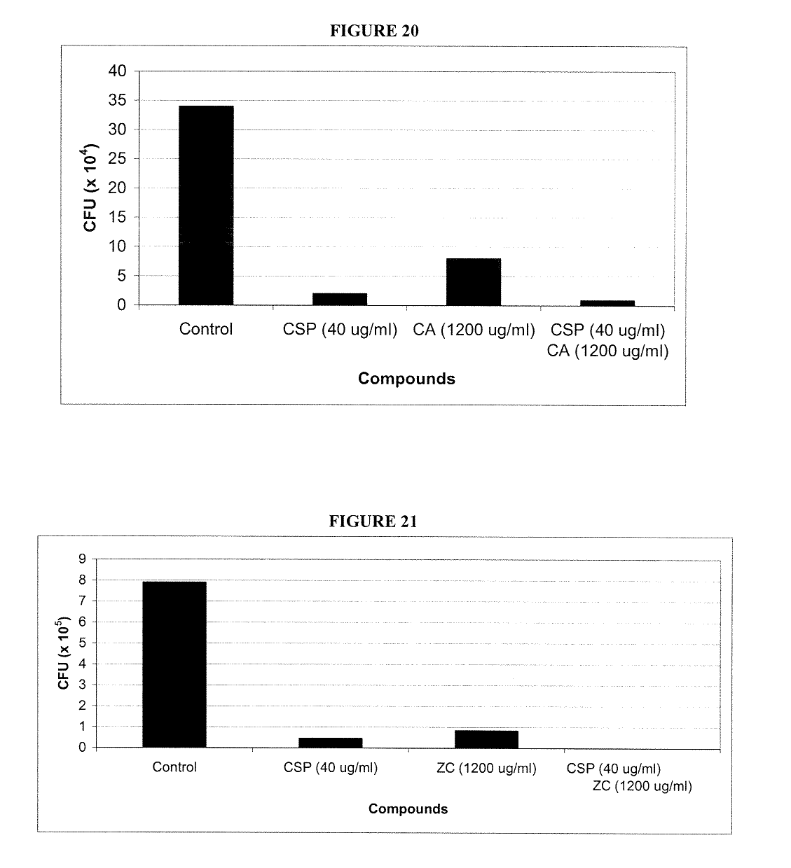

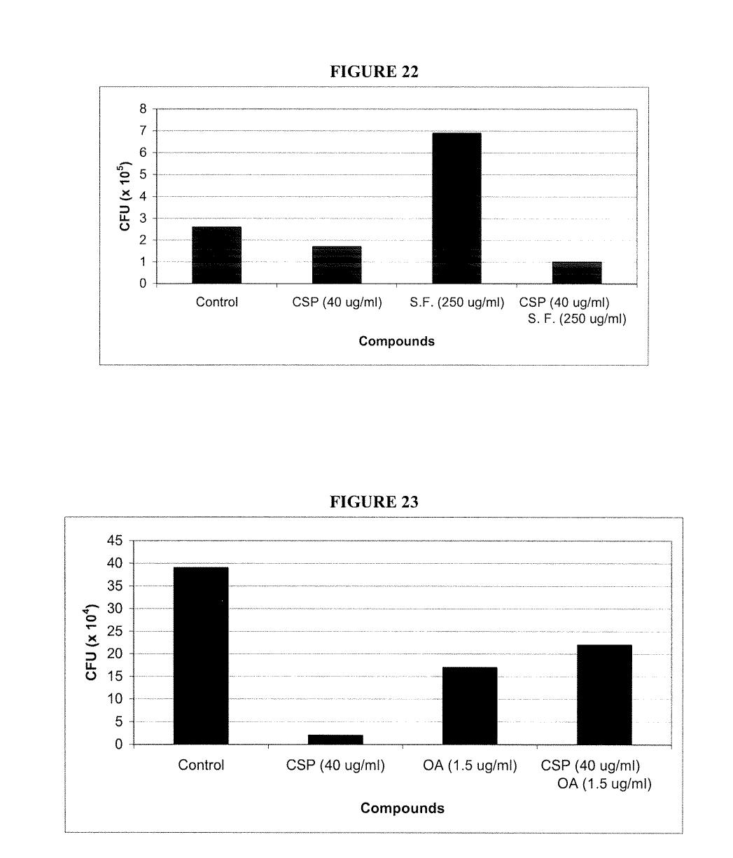

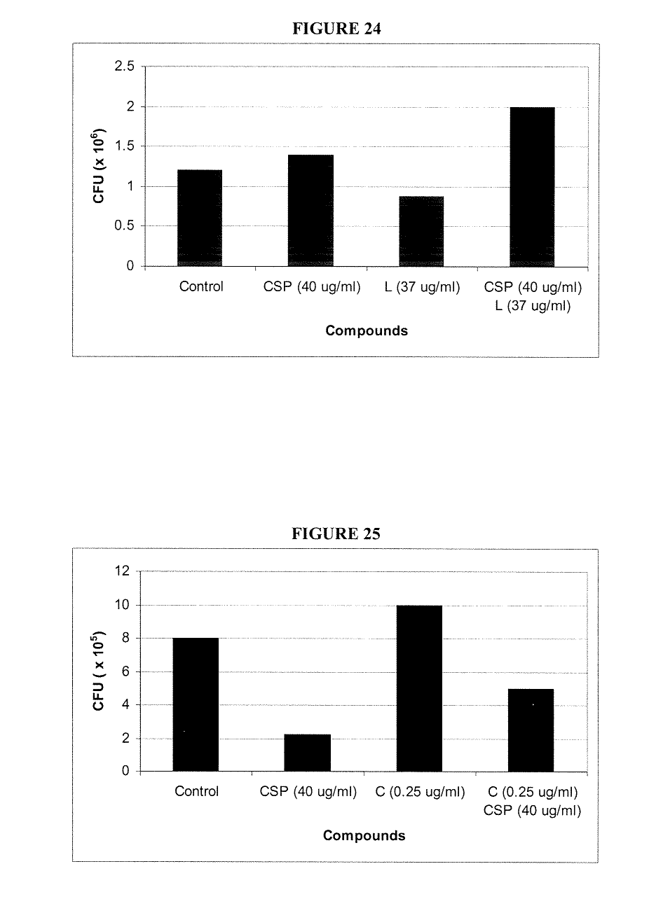

CSP in combination with the above mentioned antimicrobials and anti-caries compounds showed an enhanced inhibitory effect on the survival of biofilm-embedded S. mutans as determined by viable colony forming unit (CFU) counts (Tables 4a and 4b). The results of Tables 4a and 4b are graphically depicted in FIGS. 17-25. The combination of CSP with either nisin or triclosan or citric acid or zinc citrate or sodium fluoride had more than an additive effect in decreasing the number of CFU.

TABLE 4a
Effects of compositions comprising CSP and antimicrobial
agents on Biofilm-Embedded S. mutans¹

| Compound | Concentration (μg/ml) | CPU |
|---|---|---|
| Control | 0 | 1.6 × 10⁶ |
| CSP | 50 | 1 × 10⁶ |
| Nisin | 80 | 1 × 10⁶ |
| CSP + Nisin | | 0 |
| Control | 0 | 1.4 × 10⁶ |
| CSP | 40 | 0.1 × 10⁶ |
| Chlorhexidine | 0.15 | 2 × 10⁶ |
| CSP + Chlorhexidine | | 3.6 × 10⁶ |
| Control | 0 | 6.8 × 10⁵ |
| CSP | 40 | 7 × 10⁴ |
| Triclosan | 500 | 7 × 10² |
| CSP + Triclosan | | 0 |
| Control | 0 | 3.4 × 10⁵ |
| CSP | 40 | 2 × 10⁴ |
| Citric acid | 1200 | 8 × 10⁴ |
| CSP + Citric acid | | 1 × 10⁴ |
| Control | 0 | 8 × 10⁵ |
| CSP | 40 | 0.5 × 10⁵ |
| Zinc citrate | 1200 | 0.8 × 10⁵ |
| CSP + Zinc citrate | | 3 × 10² |
| Control | 0 | 2.6 × 10⁵ |
| CSP | 40 | 1.7 × 10⁵ |
| Sodium fluoride² | 250 | 7 × 10⁵ |
| CSP + Sodium fluoride | | 1 × 10⁵ |

¹Values represent averages of at least three determinations, CFU = colony forming units.
²An anti-caries agent

TABLE 4b
Effects of compositions comprising CSP and antimicrobial
agents on Biofilm-Embedded S. mutans¹

| Compound | Concentration (μg/ml) | CPU |
|---|---|---|
| Control | 0 | 4 × 10⁵ |
| CSP | 40 | 2 × 10⁴ |
| Oleanolic acid | 250 | 1.7 × 10⁵ |
| CSP + Oleanolic acid | | 2.2 × 10⁵ |
| Control | 0 | 1.2 × 10⁶ |
| CSP | 40 | 1.4 × 10⁶ |
| Lansoprazole | 37 | 1 × 10⁶ |
| CSP + Lansoprazole | | 2 × 10⁶ |
| Control | 0 | 7.6 × 10⁵ |
| CSP | 40 | 2.2 × 10⁵ |
| Chitosan | 0.25 | 1 × 10⁶ |
| CSP + Chitosan | | 4.5 × 10⁵ |

¹Values represent averages of at least three determinations, CFU = colony forming units.

### Example 4

### Effects of Compositions Comprising CSP and Antimicrobial Agents on the Survival of Biofllm-Embedded S. mutans on Hydroxyapatite Disks

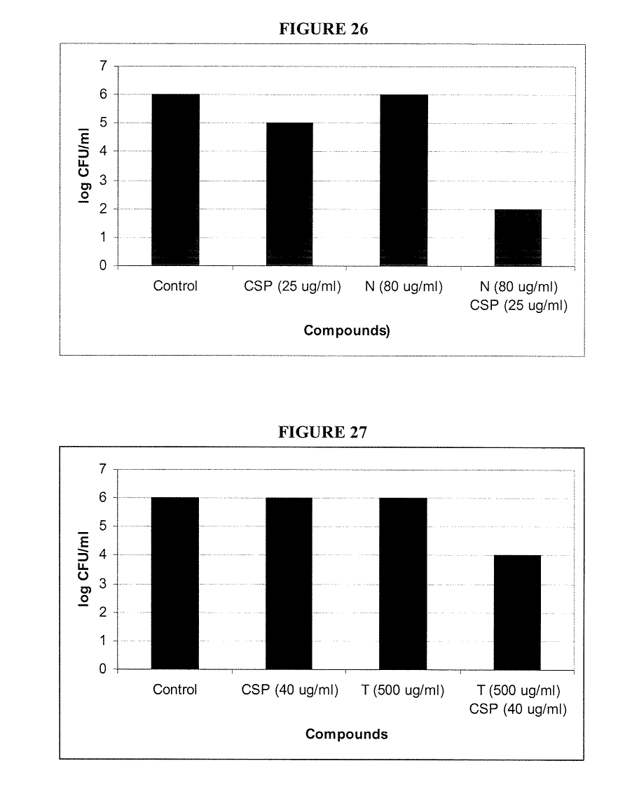

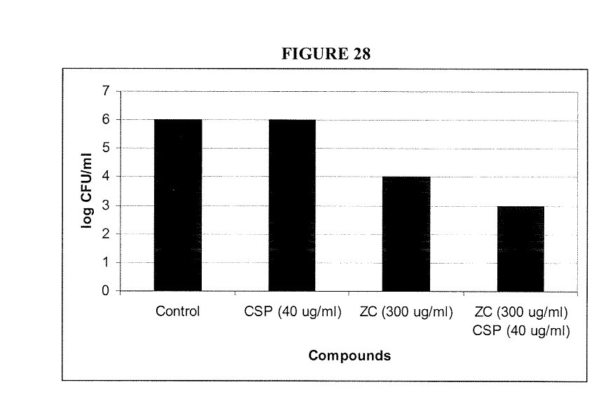

An in vitro assay was performed to determine whether CSP in combination with nisin, triclosan, zinc citrate, would show enhanced inhibitory effects on the survival of biofilm-embedded S. mutans on hydroxyapatite (HAP) disks. S. mutans biofilms were developed on HAP disks in 15 ml polystyrene test tubes to provide a rapid and simple method for assaying biofilm-embedded live oral bacteria (e.g. S. mutans) on HAP disks. A 4× diluted THYE medium supplemented with a final concentration of 0.01% hog gastric mucin (Sigma) was used as biofilm medium (BM). Formation of biofilms was initiated by inoculating 20 μl of S. mutans cell suspension (1.2×107 CFU/ml) into each of 12 tubes containing 5 ml of BM and 12 tubes were set up: three for control, three for treatment with compositions comprising synthetic CSP and nisin or triclosan or zinc citrate, three for CSP alone, and three for nisin or triclosan or zinc citrate alone. After cultures were incubated at 37° C. for 24 hours under an anaerobic condition, fluid media were removed. The HAP disks were rinsed three times with 0.9% saline solution and biofilm-embedded S. mutans cells were collected in five ml of 0.9% saline solution, gently sonicated for 30 seconds, vortexed for one minute, serially diluted, and spread on THYE plates that were incubated at 37° C. under anaerobic conditions. Biofilm-embedded viable cells were quantified by colony forming unit (CFU) counts after 48 hours of anaerobic incubation at 37° C. CSP in combination with each of the three tested compounds (nisin, triclosan, and zinc citrate) showed an enhanced inhibitory effect on the survival of biofilm-embedded S. mutans as determined by viable colony forming unit (CFU) counts (FIGS. 26-28). The combination of CSP with either nisin, triclosan, or zinc citrate had more than an additive effect in decreasing the number of CFU.

What is claimed is:

1. A composition comprising:
a) at least one peptide of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11; and
b) one or more antimicrobial agents comprising a benzimidazole, a polyol, a polyphenol, an antiseptic, an antibiotic, a bacteriocin, a citrate, an anti-caries agent, a triterpenoid, or chitosan.

2. The composition according to claim 1, wherein the peptide comprises SEQ ID NO: 2.

3. The composition according to claim 1, wherein the peptide comprises SEQ ID NO: 10.

4. The composition according to claim 1, wherein the antimicrobial agent is a benzimidazole.

5. The composition according to claim 4, wherein the benzimidizaole is lansoprazole.

6. The composition according to claim 1, wherein the antimicrobial agent is a polyol.

7. The composition according to claim 6, wherein the polyol is xylitol or sorbitol.

8. The composition according to claim 1, wherein the antimicrobial agent is a polyphenol.

9. The composition according to claim 8, wherein the polyphenol is epigallocatechin gallate.

10. The composition according to claim 1, wherein the antimicrobial agent is an antiseptic.

11. The composition according to claim 10, wherein the antiseptic is triclosan or chlorhexidine.

12. The composition according to claim 1, wherein the antimicrobial agent is a citrate.

13. The composition according to claim 12, wherein the citrate is citric acid, zinc citrate, or sodium citrate.

14. The composition according to claim 1, wherein the antimicrobial agent is a triterpenoid.

15. The composition according to claim 14, wherein the triterpenoid is oleanolic acid.

16. The composition according to claim 1, wherein the antimicrobial agent is chitosan

17. The composition according to claim 1, wherein the antimicrobial agent is a bacteriocin.

18. The composition according to claim 17, wherein the bacteriocin is nisin.

19. The composition according to claim 1 wherein the anti-caries agent is sodium fluoride.

20. The composition according to claim 1, wherein the composition comprises between about 1 μg/ml and about 100 μg/ml of the peptide.

21. The composition according to claim 1, wherein the composition comprises between 0.15 μg/ml and 15 mg/ml of an antimicrobial agent.

22. A method of inhibiting an oral biofilm comprising: administering a composition comprising at least one peptide of (a) SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11; and (b) at least one of a benzimidazole, a polyol, a polyphenol, an antiseptic, an antibiotic, a bacteriocin, a citrate, an anti-caries agent, a triterpenoid, and chitosan.

23. A method of inhibiting an oral biofilm comprising: administering a therapeutically effective amount for treating a condition caused by dental plaque associated Streptococcus mutans of a composition comprising at least one peptide of (a) SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11; and (b) at least one of a benzimidazole, a polyol, a polyphenol, an antiseptic, an antibiotic, a bacteriocin, a citrate, an anti-caries agent, a triterpenoid, and chitosan.

24. The method according to claim 23, wherein the dental plaque associated Streptococcus mutans results in dental caries, gingivitis, and endocarditis.

25. The method according to claim 22, wherein the composition comprises SEQ ID NO: 2.

26. The method according to claim 22, wherein the composition comprises SEQ ID NO: 10.

27. The method according to claim 22, wherein the antimicrobial agent is a benzimidazole.

28. The method according to claim 27, wherein the benzimidizaole is lansoprazole.

29. The method according to claim 22, wherein the antimicrobial agent is a polyol.

30. The method according to claim 29, wherein the polyol is xylitol or sorbitol.

31. The method according to claim 22, wherein the antimicrobial agent is a polyphenol.

32. The method according to claim 31, wherein the polyphenol is epigallocatechin gallate.

33. The method according to claim 22, wherein the antimicrobial agent is an antiseptic.

34. The method according to claim 33, wherein the antiseptic is triclosan or chlorhexidine.

35. The method according to claim 22, wherein the antimicrobial agent is a citrate.

36. The method according to claim 35, wherein a citrate is citric acid, zinc citrate, or sodium citrate.

37. The method according to claim 22, wherein the antimicrobial agent is a triterpenoid.

38. The method according to claim 37, wherein the triterpenoid is oleanolic acid.

39. The method according to claim 22, wherein the antimicrobial agent is chitosan.

40. The method according to claim 22, wherein the antimicrobial agent is a bacteriocin.

41. The method according to claim 40, wherein the bacteriocin is nisin.

42. The method according to claim 22, wherein the anti-caries agent is sodium fluoride.


SEQUENCE LISTING (11 sequences)

SEQ ID NO: 1 - protein, 46 aa, Streptococcus mutans
MKKTLSLKNDFKEIKTDELEIIIGGSGSLSTFFRLFNRSFTQALGK

SEQ ID NO: 2 - protein, 21 aa, Streptococcus mutans
SGSLSTFFRLFNRSFTQALGK

SEQ ID NO: 3 - protein, 18 aa, Streptococcus mutans
SGTLSTFFRLFNRSFTQA

SEQ ID NO: 4 - protein, 20 aa, Artificial
  Synthetic F1
SGSLSTFFRLFNRSFTQALK

SEQ ID NO: 5 - protein, 21 aa, Artificial
  Synthetic F2
SGSLSTFFRLFNRSFTQALGV

SEQ ID NO: 6 - protein, 20 aa, Artificial
  Synthetic H1
SGSLSTFFRLFNRSFTQLGK

SEQ ID NO: 7 - protein, 21 aa, Artificial
  Synthetic H2
SGSLSTFFVLFNVSFTQALGV

SEQ ID NO: 8 - protein, 21 aa, Artificial
  Synthetic B2
SGSLSTFFVLFNRSFTQALGK

SEQ ID NO: 9 - protein, 21 aa, Artificial
  Synthetic C2
SGSLSTFFALFNRSFTQALGK

SEQ ID NO: 10 - protein, 21 aa, Artificial
  Synthetic E2
SGSLSTFFRLFNASFTQALGK

SEQ ID NO: 11 - protein, 18 aa, Artificial
  Synthetic B3
SGTLSTFFRLFNRSFTQA